(19) United States
(12) Patent Application Publication
(10) Pub. No.: US 2025/0340959 A1
(43) Pub. Date: Nov. 6, 2025

(54) METHODS FOR DEHAIRING ANIMAL SKINS AND HIDES AND FORMULATIONS RELATED TO SAME

(71) Applicant: Buckman Laboratories International, Inc., Memphis, TN (US)

(72) Inventors: Henrique Cadete PINTO, Sao Paulo (BR); Marcelo Simoes De Almeida, Sao Paulo (BR); Erika Barbosa Neves, Sao Paulo (BR); Jesus Barguil Brasileiro, Sao Paulo (BR)

(73) Assignee: Buckman Laboratories International, Inc., Memphis, TN (US)

(21) Appl. No.: 19/189,339

(22) Filed: Apr. 25, 2025

Related U.S. Application Data
(60) Provisional application No. 63/642,021, filed on May 3, 2024.

(51) Int. Cl. C14C 1/06; C12N 9/24; C12N 9/26; C14C 1/04

(52) U.S. Cl. CPC C14C 1/065; C12N 9/2414; C12N 9/2482; C12N 9/2494; C12Y 302/01001; C12Y 302/01008; C12Y 302/01078; C14C 1/04

(57) ABSTRACT

Methods for dehairing of animal hides and skins are described. The methods can include treating the hide or skin with a dehairing formulation that includes at least one carbohydrase enzyme and treating with at least calcium hydroxide and further utilizing at least one reducing chemical during the dehairing step. A dehairing formulation is further described.

### BACKGROUND OF THE INVENTION

This application claims the benefit under 35 U.S.C. § 119 (e) of prior U.S. Provisional Patent Application No. 63/642,021 filed May 3, 2024, which is incorporated in its entirety by reference herein.

The present invention relates to the dehairing of hides and skins. The present invention further relates to dehairing formulations that can be used in dehairing processes for skins and hides.

Conventional leather processing generally involves four operations, namely, pre-tanning, tanning, post tanning, and finishing. It includes a combination of single and multi-step processes that employs as well as expels various biological, organic, and inorganic materials. Conventional beamhouse processes (liming and re-liming) employ lime and sodium sulfide and purifies the skin matrix by the removal of hair, flesh, and other unwanted materials. Various application methods include pit, paddle, drum, and painting on the flesh side. After this stage, the hide/skin can be considered a pelt. De-liming, bating, and pickling processes prepare the skin for subsequent tanning.

The traditional beamhouse processes or wet processing cleans the hides or skins and prepares them for further processing like re-tanning, fat liquoring, dyeing, and finishing. The beamhouse process includes the steps of soaking, dehairing, liming, fleshing, splitting, deliming, bating, pickling and tanning. The resulting product of these processes can be generally known as wet-blue.

Beamhouse processing, especially liming-reliming processes are known to contribute 60-70% of the total pollution load in leather processing.

The conventional lime-sulfide process can lead to the destruction of the hair, causing emissions with high chemical oxygen demand (COD), biological oxygen demand (BOD), and total suspended solid (TSS) loads in the effluent of these industries. Moreover, the use of lime in the unhairing process requires its removal, usually by the addition of ammonium salts, which represents high amounts of nitrogen in the stream of wastewater, one of the main contaminants of liquid effluents. One alternative to conventional lime-sulfide process is enzymatic unhairing and the utilized enzyme preparations, but currently almost all are done by proteases sometimes in association with lipases. On the other hand, the main difficulty which hinders the utilization of protease in unhairing is to confine the proteolytic activity to the epidermis layer, hair follicles/hair and basement membrane. However, the extension of proteolytic activity towards collagen structure, to some degree, is inevitable, thus impairing the properties of leather. At present, the high control of proteolysis is essential to enzymatic unhairing by protease but such control has not been obtained on a commercial level and on a consistent basis.

Accordingly, a need exist that would permit effective dehairing of skins and hides, especially on a commercial level using an enzyme treatment, but which confines the enzymatic activity to the epidermis layer, hair follicles/hair and basement membrane and thus preserve the integrity of the grain of the skin and hide. A need further exists for enzyme treatments that can achieve effective dehairing of the skins and hides without damage to the collagen structure and leather properties.

### SUMMARY OF THE INVENTION

A feature of the present invention is to provide a dehairing process that can improve the removal of hair, hair roots, and/or epidermis keratin.

Another feature of the present invention is to provide a dehairing process that can provide effective dehairing of the skins and hides and also improve the leather grain quality.

An additional feature of the present invention is to provide a dehairing process that can use enzymatic treatment but in the absence of protease enzymes.

Also, a feature of the present invention is to provide a dehairing process that can reduce the amount of lime utilized in the dehairing process.

A further feature of the present invention is to provide a dehairing process that can reduce the amount of time for effective dehairing, especially utilizing an enzyme treatment.

An additional feature of the present invention is to provide an enzymatic dehairing process that can confine the enzyme activity to the epidermis layer, hair follicles/hair, and/or basement membrane of the skins and hides.

A further feature of the present invention is to provide an enzymatic dehairing process that can greatly diminish or avoid the extension of enzyme or proteolytic activity towards the collagen structure of the skins and hides.

Y et a further feature of the present invention is to provide a dehairing process that can provide a dehaired skin or hide that has well defined pores and/or flatter grains and/or have improved characteristics of better cleaning and/or better uniformity of the softness of the pelt and in the final leather, and obtain a clearer pelt, which provides a more vibrant and homogeneous dyeing of the pelt, and which can improve the process with some reduction in bleaching agents.

To achieve these and other advantages and in accordance with the purposes of the present invention, as embodied and broadly described herein, the present invention relates to a method for dehairing animal skins or hides. The method includes soaking animal skins or hides having skin hair in an aqueous soaking solution to wet the animal skins or hides having skin hair and obtain wet skins or hides. The method further includes contacting the wet skins or hides with a dehairing formulation and also calcium hydroxide for a sufficient time so as to decompose at least a majority of the skin hair present on the wet skins or hides. The method further includes contacting the wet skins or hides while the dehairing formulation is present with at least one reducing chemical. The method then includes recovering the animal skins or hides, for instance for further processing using conventional post-dehairing steps. The dehairing formulation can include or comprise at least one carbohydrase enzyme. The dehairing formulation can optionally include or comprise at least one lipase enzyme. The dehairing formulation can optionally further include or comprise at least one non-ionic surfactant and/or at least one divalent salt.

The present invention also relates to a dehairing formulation that includes from about 10 wt % to 90 wt % carbohydrase enzyme, from about 2 wt % to about 35 wt % or from about 2 wt % to 20 wt % non-ionic surfactant; and from about 0.1 wt % to 1 wt % divalent salt, and optionally from about 10 wt % to about 70 wt % solvent, all based on total weight of the dehairing formulation.

The present invention also relates to a dehairing formulation that includes from about 10 wt % to 90 wt % carbohydrase enzyme, from about 0.1 wt % to about 50 wt % or more of a lipase enzyme, from about 2 wt % to about 35 wt % or from about 2 wt % to 20 wt % non-ionic surfactant; and from about 0.1 wt % to 1 wt % divalent salt, and optionally from about 10 wt % to about 70 wt % solvent, all based on total weight of the dehairing formulation.

Additional features and advantages of the present invention will be set forth in part in the description which follows, and in part will be apparent from the description, or may be learned by practice of the present invention. The objectives and other advantages of the present invention will be realized and obtained by means of the elements and combinations particularly pointed out in the written description and appended claims.

As used herein, the terms “hide,” “animal hide,” “skin,” and “animal skin” are all used interchangeably to refer to the flayed or stripped skin or outer layer of an animal, particularly of an animal whose skin is useful for converting into leather. The skin can be ovine skins, porcine skins, bovine hides, or caprine skins. Examples of animals from which skin can be taken to make leather include, but are not limited to: cattle, pigs, deer, kangaroos, goats, camels, sheep, horses, alligators, crocodiles, snakes, birds, seals, eel, fish and walrus.

As used herein, the terms “wet, tanned hide,” “wet, tanned skin,” “tanned hide,” and “tanned skin” are all used interchangeably to refer to a hide or skin that has been modified by reaction with any tanning agent, to yield a stable, partially processed, intermediate or finished product that usually does not putrefy under normal storage conditions and will withstand exposure to heat without a significant deterioration as long as the shrinkage temperature is not exceeded. The reference to “wet” does mean a product that is not a finished dry leather product that a consumer would buy and use, and the “wet” refers to a hide or skin in a wet or moist state prior to being formed/dried into crust leather.

It is to be understood that both the foregoing general description and the following detailed description are exemplary and explanatory only and are only intended to provide a further explanation of the present invention, as claimed.

### DETAILED DESCRIPTION OF THE PRESENT INVENTION

The present invention provides methods for the dehairing of hides and skins. The present invention further relates to dehairing formulations that can be used in dehairing processes for skins and hides. As explained herein, various advantages and improvements are achieved with the present invention.

In more detail, the present invention, in part, relates to a method for dehairing animal skins or hides or both. The method comprises, consists essentially of, consists of, or includes the following steps. One step in the method involves soaking animal skins or hides having skin hair (on them) in an aqueous soaking solution to wet the animal skins or hides having skin hair and obtain wet skins or hides. If the animal skins or hides to be processed are already soaked beforehand, this step can be skipped.

The method further involves the step of contacting the wet skins or hides with a dehairing formulation and calcium hydroxide for a sufficient time so as to decompose at least a majority of the skin hair present on the wet skins or hides.

The method further includes the step of contacting the wet skins or hides while the dehairing formulation is present with at least one reducing chemical.

The method further includes the step of recovering the animal skins or hides after a majority of the skin hair is removed.

The dehairing formulation comprises, consists essentially of, consists of, or includes at least one carbohydrase enzyme.

As an option, the dehairing formulation further comprises or includes at least one lipase enzyme.

As an option, the dehairing formulation further comprises or includes at least one non-ionic surfactant and/or at least one divalent salt. Preferably both at least one non-ionic surfactant and at least one divalent salt are used.

For purposes of the present invention, the dehairing formulation means a pre-formed formulation, and/or one that forms in-situ by separate addition of each component, and/or is the presence of a) at least one carbohydrase enzyme, b) optionally at least one lipase enzyme, c) optionally at least one non-ionic surfactant, d) optionally at least one divalent salt and/or e) any other optional component that may be part of the formulation, amongst the animal skin(s) and/or hide(s) and/or the aqueous soaking solution.

In yet further detail, the various aspects of the methods of the present invention and the dehairing formulation are provided.

Regarding the soaking step, the soaking step can comprise of one or several separate soaking steps. The soaking step can occur in a container or drum or tank or tumbler, optionally with a device(s) for agitation (collectively these can be referred to as a drum or float). The same container or drum or a different one can be used when the dehairing formulation contacts the wet skins or hides.

When several soaking steps are used, one can be considered a pre-soak. The soaking liquid used can be water alone. The soaking liquid, as an option, can be water used with one or more enzymes, tenside, one or more surfactants, one or more solvents, and/or one or more preservation agents, one or more detergents, and/or soda. Conventional techniques and conventional amounts of additives can be used for the soaking step.

The soaking step serves to clean adhering dirt from the raw hide and skins, and/or to remove curing salt and other preserving agents from the hides and skins, and/or to dissolve out water soluble protein components at least partially, and/or to return the hides and/or skins to the degree of swelling which they had in their original condition but was lost in the course of the curing process. Besides water, the soaking of skins and hides can be accomplished with the use of surfactants, and/or amines and/or organic solvents and/or enzymes, such as protease and/or lipase.

The soaking can be conducted utilizing a paddle, drum, or mixer as mechanical agitation to accelerate the soaking process. The hides or skins can be soaked in a drum with a float length of 250% (or within 50% of this, for example) and sheep skins especially for wool-on are soaked in paddle with float length of 2000% (or within 50% of this, for example). The soak float is generally discarded on conclusion of the soak. Subsequent to the soak, the hides or skins can be worked up including being conveyed to the dehairing operation.

A soaking process of the present invention may be performed at conventional soaking conditions, i.e. the pH of soak float in the range pH 4-12, for example, in the range pH 6-10, or the range pH 7-8; and for instance, at a temperature in the range 5-65 C, such as 15-45 C, and a soak time, for instance, in the range of 1-48 hours, such as 2-24 hours. The presoaking can be simpler and shorter, for instance, performing the soak with water at the same temperature, alternatively a small amount of tenside is added to the water in the same vessel for 0.5 to 5 hours.

The soaking step (whether one or multiple steps) can be for at least 30 minutes, or at least 1 hour, or at least 5 hours, or at least 10 hours, or at least 15 hours, or at least 20 hours. The soaking time can be from about 1 hour to about 20 hours or more.

As an option, the soaking step(s) can be wherein the soaking is in the absence of any enzyme.

As an option, the soaking step(s) can be wherein the soaking is in the absence of any carbohydrase enzyme.

As an option, the soaking step(s) can be wherein the soaking is in the absence of any lipase enzyme.

As an option, the soaking step(s) can be wherein the soaking is in the absence of any

protease enzyme.

As an option, the soaking step(s) can be wherein the soaking is in the absence of any carbohydrase enzyme and protease enzyme.

As an option, the soaking step(s) can be wherein the soaking is in the absence of any carbohydrase enzyme, lipase enzyme, and protease enzyme.

In the soaking step, prior to the dehairing step with the dehairing formulation, as an option, the soaking step removes no or essentially no skin hair (e.g., less than 10% by surface area of any hair present or less than 5% by surface area or less than 1% by surface area or less than 0.1% by surface area) from the animal hides or skins.

Regarding the step of contacting of the wet skins or hides with the dehairing formulation, this ‘contacting’ can be accomplished in any manner that permits the dehairing formulation to coat or fully envelop or fully contact the skin or hide such that the hair on the skins or hides is at least in part dissolved or completely dissolved during the contact time with the dehairing formulation.

One way the contacting with the dehairing formulation can be achieved is by floating the wet skins or hides in a bath in a drum, as described earlier, that contains at least the dehairing formulation and water. This operation can be considered floating of the skins or hides with the dehairing formulation. The term “floating” is a term of art understand in the industry. Generally, the skins or hides will be introduced into a drum or similar container along with the dehairing formulation and water and the drum rotated about the cylindrical axis of the drum. The drums can be any size. Typical sizes for these drums are from about 1,000 kg to about 20,000 kg for high capacity, with respect to the weight of the components/formulation in the drum. Capacities above or below these amounts can be used.

For instance, water can be added in an amount that provides satisfactory rubbing between the individual hides or skins, and the mechanical influence are usually ensured by drumming the skin and hides e.g. in a beam house drummer or the like.

As an example, water can be added in an amount of from about 50% to about 200% in relation to the dry weight of the hides or skins, such as from about 70% to about 120% thereof.

During the dehairing operation, the removed hairs or dissolved hairs or partially dissolved hairs can be removed from the dehairing bath, for instance by continuous or semi-continuous filtration during the dehairing step.

As an option, prior to the dehairing step, the skins or hides can be subjected to a green fleshing step. The green fleshing is performed to remove fat which may interfere with the dehairing.

As an option, after the dehairing step, a fleshing step can be performed on the dehaired skins and/or hides.

The dehairing formulation can be present in the bath in an amount of from about 0.1 wt % to about 1 wt % or more, based on the total wt % of the animal skins or hides prior to the soaking step. This amount can be from 0.1 wt % to 1 wt %, or from 0.2 wt % to 1 wt %, or 0.3 wt % to 1 wt %, or from 0.4 wt % to 1 wt %, or from 0.5 wt % to 1 wt %, or from 0.6 wt % to 1 wt %, or from 0.7 wt % to 1 wt % or more, or from 0.1 wt % to 0.9 wt %, or from 0.1 wt % to 0.8 wt %, or from 0.1 wt % to 0.7 wt %, or from 0.1 wt % to 0.6 wt %, or from 0.1 wt % to 0.5 wt %, or any range based upon any two values described herein.

As an option, the dehairing formulation can be in the absence of any protease enzyme. Preferably no protease enzyme is present or substantially not present (e.g., less than 100 ppm or less than 10 ppm or less than 1 ppm or zero in the dehairing formulation as a concentrate). As an option, protease enzyme is absent from the aqueous soaking solution and/or the hides and/or skins and/or any tank or container that is used during the methods of the present invention.

As an option, the dehairing formulation can be in concentrate form. When a concentrate, the dehairing formulation can comprise from about 10 wt % to 90 wt % carbohydrase enzyme, based on total weight of the dehairing formulation. This amount can be from 10 wt % to 90 wt %, from 15 wt % to 90 wt %, from 20 wt % to 90 wt %, from 30 wt % to 90 wt %, from 40 wt % to 90 wt %, from 50 wt % to 90 wt %, from 60 wt % to 90 wt %, from 70 wt % to 90 wt %, from 80 wt % to 90 wt %, from 10 wt % to 85 wt %, from 10 wt % to 80 wt %, from 10 wt % to 75 wt %, from 10 wt % to 70 wt %, from 10 wt % to 65 wt %, from 10 wt % to 60 wt %, from 10 wt % to 55 wt %, from 10 wt % to 50 wt %, from 10 wt % to 45 wt %, from 10 wt % to 40 wt %, from 10 wt % to 35 wt %, from 10 wt % to 30 wt %, from 10 wt % to 25 wt %, from 10 wt % to 20 wt %, or any range based upon any two values described herein.

The at least one carbohydrase enzyme can be, for instance, a xylanase, a mannanase, a cellulase, and/or an amylase.

The carbohydrase enzyme can have any effective specific enzyme activity. For instance, the carbohydrase enzyme can have a specific enzyme activity of from about 1,000 to 100,000 or more, where the specific enzyme activity is based on an enzyme per milligram of total protein, expressed in μmol min⁻¹ mg⁻¹. This activity can be from 1,000 to 100,000, from 1,000 to 75,000, from 1,000 to 50,000, from 1,000 to 25,000, from 1,000 to 15,000, from 1,000 to 10,000, from 1,000 to 5,000, from 1,000 to 2,500 expressed in μmol min⁻¹ mg⁻¹.

The carbohydrase enzyme can consist of one or more microbial enzymes. As an option, no non-microbial enzymes are present as the carbohydrase.

As an option, no non-microbial enzyme is present at any stage of the method of the present invention.

The carbohydrase enzyme can be any hydrolase belonging to the enzyme sub-class EC 3.2.1. Examples include, but are not limited to, enzymes hydrolyzing O- and S-glycosyl compounds. The carbohydrase can be alpha-1,4-carbohydrase which can hydrolyze alpha 1-4 carbohydrate linkages, e.g. alpha-amylase, amyloglucosidase, maltase and so forth; and beta-1,4-carbohydrase which can hydrolyze beta 1-4 carbohydrate linkages, e.g. xylanase, mannanase, mannosidase, beta-glucanases, cellulases and so forth. The term “EC” (Enzyme Class) refers to the internationally recognized enzyme classification system, Recommendations (1992) of the Nomenclature Committee of the International Union of Biochemistry and Molecular Biology, Academic Press, Inc., 1992.

The carbohydrase can be a mannanase, which are hemicellulases classified as EC 3.2.1.78, and called endo-1,4-beta-mannosidase. Mannanase can for example be beta-mannanase, endo-1, 4-mannanase, and galacto-mannanase. Mannanase can be capable of catalyzing the hydrolysis of 1,4-beta-D-mannosidic linkages in mannans, including glucomannans, galactomannans and galactoglucomannans. The mannanase can be polysaccharides primarily or entirely composed of D-mannose units. The mannanase can be alkali tolerant. The mannanase may be of any origin such as a bacterium or a fungal organism, or chemically or genetically modified mutants (variants). The mannanase can be derived from a strain of the filamentous fungus genus Aspergillus, such as Aspergillus niger or Aspergillus aculeatus.

Mannanases have been identified in several Bacillus organisms. For example, Talbot et al., Appl. Environ. Microbiol., Vol. 56, No. 11, pp. 3505-3510 (1990) describes a beta-mannanase derived from Bacillus stearothermophilus. Mendoza et al., World J. Microbiol. Biotech., Vol. 10, No. 5, pp. 551-555 (1994) describes a beta-mannanase derived from Bacillus subtilis. JP-A-03047076 discloses a beta-mannanase derived from Bacillus sp. JP-A-63056289 describes the production of an alkaline, thermostable beta-mannanase. JP-A-63036775 relates to the Bacillus microorganism FERM P-8856 which produces beta-mannanase and beta-mannosidase. J P-A-08051975 shows alkaline beta-mannanases from alkalophilic Bacillus sp. AM-001. A purified mannanase from Bacillus amyloliquefaciens is shown in WO 97/11164. WO 91/18974 describes a hemicellulase such as a glucanase, xylanase or mannanase active. Examples of commercially available mannanases include Mannaway™ available from Novozymes A/S Denmark and Mannastar™ available from Genencor International Inc USA.

The carbohydrase enzyme can be a xylanase. The xylanase can be any member of the glycoside hydrolase families (EC 3.2.1.8) which have endo-1,4-β-xylanase activity. The beta-xylanase can be alkali tolerant. Suitable beta-xylanases include those of bacterial or fungal origin, or chemically or genetically modified mutants (variants).

Fungal xylanases may be derived from strains of genera including Aspergillus, Disporotrichum, Penicillium, Neurospora, Fusarium and Trichoderma. Examples of suitable xylanases include xylanases derived from H. insolens (WO 92/17573); Aspergillus tubigensis (WO 92/01793); A. niger (Shei et al., 1985, Biotech and Bioeng. Vol. XXVII, pp. 533-538, and Fournier et al., 1985, Biotech. Bioeng. Vol. XXVII, pp. 539-546; WO 91/19782 and EP 463 706); A. aculeatus (WO 94/21785).

Examples of suitable bacterial xylanases include xylanases derived from a strain of Bacillus, such as Bacillus subtilis, such as the one shown in U.S. Pat. No. 5,306,633 or Bacillus agaradhaerens, including Bacillus agaradhaerens AC13 shown in WO 94/01532, a strain of Bacillus pumilus, such as the one shown in WO 95/182109, a strain derived from of Bacillus stearothermophilus, such as the one shown in WO 95/182109.

Commercially available xylanases include SHEARZYME™, BIOFEED WHEAT™, PULPZYME™ HC (from Novozymes A/S), BioBrite™ EB (from Logen, Canada) and SPE-ZYME™ CP (from Genencor International Inc USA).

The carbohydrase enzyme can be an amylase. Any alpha-amylase (EC. 3.2.1.1) can be used, for instance, which catalyzes the hydrolysis of starch and other linear and branched 1,4-glucosidic oligo-and polysaccharides. The alpha-amylase can be an alkali alpha-amylase. Suitable alpha-amylases include those of bacterial or fungal origin. Chemically or genetically modified mutants (variants) are included. The alpha-amylase can include a carbohydrate-binding module (CBM) as defined in WO 2005/003311, such as in a family 20 CBM as defined in WO 2005/003311. The fungal alpha-amylase can be of yeast or filamentous fungus origin. The alpha-amylases can include, for example, alpha-amylases obtainable from Aspergillus species, in particular from Aspergillus niger, A. oryzae, and A. awamori, A. kawachii, such as the acid alpha-amylase disclosed as SWISSPROT P56271, or described in more detail in WO 89/01969 (Example 3).

The alpha-amylase can be of bacterial origin. The bacterial alpha-amylase can be derived from a strain of Bacillus, such as Bacillus licheniformis, Bacillus amyloliquefaciens, Bacillus stearothermophilus, Bacillus subtilis, or other Bacillus sp., such as Bacillus sp. NCIB 12289, NCIB 12512 (WO 95/26397), NCIB 12513 (WO 95/26397), DSM 9375 (WO 95/26397), DSM Z 12648 (WO 00/60060), DSMZ 12649 (WO 00/60060), KSM AP1378 (WO 97/00324), KSM K 36 or KSM K38 (EP 1022334). The enzyme can be a Bacillus sp. alpha-amylases as shown in WO 95/26397 as SEQ ID NOS. 1 and 2, respectively, the A A 560 alpha-amylase as shown as SEQ ID NO: 2 in WO 00/60060. The Bacillus licheniformis alpha amylase can be SEQ ID NO: 2 as shown in WO 96/23874.

The bacterial alpha-amylase can be the SP722 alpha-amylase shown as SEQ ID NO: 2 in WO 95/26397 or the A A 560 alpha-amylase. The alpha-amylase can be or include the polypeptide of SEQ ID NO: 1 from Bacillus licheniformis, and the polypeptide of SEQ ID NO:4 from Bacillus sp., and polypeptides having at least 80% identity, or 90% identity, or 95% identity with SEQ ID NO: 1 or SEQ ID NO:4. The alpha-amylase can be differing from the parent protein of SEQ ID NO: 1 or SEQ ID NO:4, by insertion, deletion and/or substitution of one or several amino acid residues, such as 1, 2, 3, 4, 5, 10, 15, 20, or 30 amino acid residues.

Commercially available alpha-amylase products or products comprising alpha-amylases include products sold under the following tradenames: Termamyl™, Aquazym™ Ultra, Aquazym™, NATALASE™, STAINZYME™ (Novozymes A/S, Denmark), Bioamylase-D (G), BIOAMYLASE™ L (Biocon India Ltd.), KEMZYM™ AT 9000 (Biozym GmbH, Austria), PURASTAR™ ST, PURASTAR™ HPAmL, PURAFECT™ OxAm, RAPIDASE™ TEX (Genencor Int. Inc, USA), KAM (KAO, Japan).

The carbohydrase enzyme can be present in the dehairing formulation in an amount of from about 10 wt % to about 90 wt %, or from about 10 wt % to 80 wt %, or from about 10 wt % to 70 wt %, or from about 10 wt % to 60 wt %, or from 10 wt % to 50 wt %, or from 10 wt % to 40 wt %, or from 10 wt % to 30 wt %, or from 20 wt % to 90 wt %, or from 20 wt % to 90 wt %, or from 30 wt % to 90 wt %, or from 40 wt % to 90 wt %, or from 50 wt % to 90 wt %, or from 60 wt % to 90 wt %, or from 70 wt % to 90 wt %, or any range based upon any two values described herein, based on total weight of the dehairing formulation.

As indicated, as an option, at least one lipase can be present in the dehairing formulation. A ny suitable lipase may be used in the methods of the present invention. One or more lipases can be used. If more than one type is used, the lipases can be introduced together or separately at the same or different locations.

Lipases can include the enzymes classified by EC 3.1.1.3. Reference is made to the Recommendations (1992) of the Nomenclature Committee of the international Union of Biochemistry and Molecular Biology, Academic Press Inc., 1992. Lipase can be derived or isolated from various fungi and/or bacteria, and/or other microorganisms, or from pancreatic sources (e.g., pancreatic lipase). As an option, the lipase can be of microbial origin, in particular of bacterial, fungal, or yeast origin. The lipase can be derived from any source, including, for example, a strain of Aspergillus, a strain of Achromobacter, a strain of Bacillus, a strain of Candida, a strain of Chromobacter, a strain of Fusarium, a strain of Humicola, a strain of Hyphozyma, a strain of Pseudomonas a strain of Rhizomucor, a strain of Rhizopus, or a strain of Thermomyces, or any combinations thereof.

Examples of lipases include, but are not limited to, triacyl glycerol lipase (TAG lipase), triacylglycerol acylhydrolase lipase, or combination thereof. Lipases can include the lipases described, for example, in U.S. Pat. Nos. 6,074,863; 5,507,952; and 5,356,800 and in U.S. Patent Application Publication Nos. 2009/0065159 A 1 and 2002/0137655 A 1, which are incorporated in their entireties by reference herein. Commercially available products containing lipase can be used. Commercial lipases which can be used, for example, include Candida antarctica lipase A, Candida antarctica lipase 8, Resinase A2X, Resinase NT, Resinase HT or NovoCor® ADL (available from Novozymes A/S), Greasex 50L, PALATASE™ A, PALATASE™ M (available from Novo Nordisk), Pancreatic Lipase 250 (available from Miles Laboratories, Inc.), Lipase G-1000 (available from Genencor), and Optimyze®, Buzyme® 2515, and Buzyme® 2517 (available from Buckman Laboratories International, Inc.).

Alkaline lipases can be used. Commercial liquid enzymatic compositions containing lipases are available under the names Lipolase 100, Greasex 50L, Palatase™ A, Palatase™ M, and nipozyme™, which are all supplied by Novo Nordisk.

The lipase used in the present invention can have any positive amount of activity. For instance, the activity can be at least about 5.0 LU/Gm/min, such as at least about 10 LU/Gm/min, or at least about 15 LU/Gm/min, for instance from about 15.0 to about 30.0 LU/Gm/min defined as lipase units per gram per minute (LU/gm/min). The lipases which can have this activity can be, for instance, triacylglycerol lipase or other lipases. For instance, Resinase A2X lipase from Novozyme has activity of about 15.0 to about 20.0 LU/Gm/min defined as lipase units per gram per minute (LU/gm/min).

The lipase enzyme, if present, can have any effective specific enzyme activity. For instance, the lipase enzyme can have a specific enzyme activity of from about 1,000 to 100,000 or more, where the specific enzyme activity is based on an enzyme per milligram of total protein, expressed in μmol min⁻¹ mg⁻¹. This activity can be from 1,000 to 100,000, from 1,000 to 75,000, from 1,000 to 50,000, from 1,000 to 25,000, from 1,000 to 15,000, from 1,000 to 10,000, from 1,000 to 5,000, from 1,000 to 2,500 expressed in μmol min⁻¹ mg⁻¹.

It is to be understood that the term lipase, can encompass wild-type lipase enzymes, as well as any variant thereof that retains the activity in question, such as chemically modified or protein engineered mutants. Such variants may be produced by recombinant techniques. The wild-type lipase enzymes may also be produced by recombinant techniques, or by isolation and purification from the natural source.

The lipase enzyme can be present in the dehairing formulation in an amount of from about 0.1 wt % to about 50 wt % or more, or from about 0.1 wt % to 45 wt %, or from about 0.1 wt % to 40 wt %, or from about 0.1 wt % to 35 wt %, or from 0.1 wt % to 30 wt %, or from 0.1 wt % to 25 wt %, or from 0.1 wt % to 20 wt %, or from 0.5 wt % to 50 wt %, or from 1 wt % to 50 wt %, or from 5 wt % to 50 wt %, or from 10 wt % to 50 wt %, or from 15 wt % to 50 wt %, or from 20 wt % to 50 wt %, or from 25 wt % to 50 wt %, or any range based upon any two values described herein, based on total weight of the dehairing formulation.

The lipase enzyme can consist of one or more microbial enzymes. As an option, no non-microbial enzymes are present as the lipase.

When multiple enzymes are present (either one or more carbohydrase enzymes and/or carbohydrase enzyme(s) in combination with one or more lipase enzymes, the enzymes can be added as part of a pre-mixture, added separately, or added in any order in forming the dehairing formulation or in introducing the formulation (or components thereof) to the or contacting with the wet skins or hides.

In general, the temperature and pH of the wet skins and hides and/or the aqueous soaking solution into which the enzymes are introduced should not be deactivating with respect to the enzyme(s).

For any enzyme used herein, the enzyme can be considered to have mesophilic enzyme activity (e.g., the carbohydrase enzyme can be a mesophile carbohydrase enzyme, and/or the lipase enzyme can be a mesophile lipase enzyme). The enzyme can be considered to be a mesophile enzyme.

For any enzyme used herein, the enzyme can be considered to have thermophilic enzyme activity (e.g., the enzyme has enzyme activity at temperatures in the range of from 10 deg C. to 80 deg C.). The enzyme can be considered to be a thermophile enzyme (e.g., the carbohydrase enzyme can be a thermophile carbohydrase enzyme, and/or the lipase enzyme can be a thermophile lipase enzyme).

When a carbohydrase enzyme(s) and a lipase enzyme(s) are both present in the dehairing formulation, the weight ratio of carbohydrase enzyme to lipase enzyme (based on the weight of the dehairing formulation) can be 3:1, 2.75:1, 2.5:1, 2.25:1, 2:1, 1.75:1, 1.5:1, 1.25:1 or 1:1 (carbohydrase enzyme: lipase enzyme). Other weight ratios above or below any of these ratios is also an option. As an option, when the carbohydrase enzyme(s) and the lipase enzyme(s) are both present in the dehairing formulation, the weight percent of the carbohydrase enzyme can be higher than the weight percent of the lipase enzyme amount. The above amounts and ratios, as an alternative and option, can be based on an enzyme volume basis.

As an option, the dehairing formulation, optionally as a concentrate, comprises from about 10 wt % to 90 wt % carbohydrase enzyme, optionally from about 0.1 wt % to 50 wt % lipase enzyme, from about 2 wt % to about 35 wt %, or from about 2 wt % to 20 wt %, or from about 5 wt % to about 35 wt % non-ionic surfactant; and from about 0.1 wt % to 1 wt % divalent salt, based on total weight of the dehairing formulation. The amount of the carbohydrase enzyme can be any of the amounts described above. The amount of the lipase enzyme can be any of the amounts described above.

With respect to the non-ionic surfactant, examples include, but are not limited to, one or more selected from: oleo (fatty acid) esters, oleo alkoxylates, oleo fatty acid oils, alcohol alkoxylates, EO-PO block copolymers, and/or any combinations thereof or any blends thereof.

More specific examples of the non-ionic surfactants include the following:

| | |
|---|---|
| OLEO (FATTY ACID) ESTERS | PEG 400 DO |
| OLEO (FATTY ACID) ESTERS | PEG 600 DIOLEATE |
| OLEO (FATTY ACID) ESTERS | PEG 600 |
| | MONOOLEATE |
| OLEO | T DET C-40 |
| ETHOXYLATES/ALKOXYLATES | |
| OLEO-FATTYACID, OILS, | LUMULSE GM O- |
| GLYCERIN | K (GLYCEROL |
| | OLEATE) |
| OLEO (FATTY ACID) ESTERS | PEG 400 DO |
| OLEO (FATTY ACID) ESTERS | PEG 400 DO |
| OLEO (FATTY ACID) ESTERS | PEG 400 DO |
| OLEO (FATTY ACID) ESTERS | PEGOSPERSE 400M O |
| OLEO (FATTY ACID) ESTERS | PEGOSPERSE 400M O |
| OLEO (FATTY ACID) ESTERS | PEG 600 DIOLEATE |
| OLEO (FATTY ACID) ESTERS | PEG 600 |
| | MONOOLEATE |
| OLEO (FATTY ACID) ESTERS | PEG 600 MONOESTER |
| | OF TOFA |
| OLEO (FATTY ACID) ESTERS | PEG 600 DIOLEATE |
| OLEO (FATTY ACID) ESTERS | PEG 600 |
| | MONOOLEATE |
| OLEO-FATTYACID, OILS, | SOY BEAN OIL FATTY |
| GLYCERIN | ACIDS |
| OLEO | T DET C-40 |
| ETHOXYLATES/ALKOXYLATES | |
| OLEO | T DET C-40 |
| ETHOXYLATES/ALKOXYLATES | |
| OLEO (FATTY ACID) ESTERS | PEG 400 monoester of |
| | TOFA |
| OLEO | T DET C-40 |
| ETHOXYLATES/ALKOXYLATES | |
| SURFACT/DEF (ALCOHOL | ALFONIC TDA-9 |
| ALKOXYLATES) | |
| SURFACT/DEF (ALCOHOL | ALFONIC TDA-9 |
| ALKOXYLATES) | |
| SURFACT/DEF (ALCOHOL | 19253 |
| ALKOXYLATES) | |
| SURFACT/DEF (ALCOHOL | 19253 |
| ALKOXYLATES) | |
| SURFACT/DEF (ALCOHOL | 19253 |
| ALKOXYLATES) | |
| SURFACT/DEF (ALCOHOL | SF-9 |
| ALKOXYLATES) | |
| SURFACTANT (BLEND OR | ECOPOL 6660 |
| OTHER) | |
| SURFACT/DEF (ALCOHOL | ISO-TDA-30 (70%) |
| ALKOXYLATES) | |
| SURFACT/DEF (ALCOHOL | ISO-TDA-30 (70%) |
| ALKOXYLATES) | |
| SURFACT/DEF (ALCOHOL | ISO-TDA-30 (70%) |
| ALKOXYLATES) | |
| SURFACTANT (BLEND OR | EMULSOGEN IT |
| OTHER) | |
| SURFACTANT (BLEND OR | EMULSOGEN ITS |
| OTHER) | |
| SURFACT/DEF (EO-PO BLOCK | EO/PO BLOCK |
| COP) | COPOLYMER BPR2 |
| SURFACT/DEF (EO-PO BLOCK | EO/PO BLOCK |
| COP) | COPOLYMER EPO-61 |
| SURFACT/DEF (EO-PO BLOCK | EO/PO BLOCK |
| COP) | COPOLYMER L-62 |
| SURFACT/DEF (EO-PO BLOCK | 25R 2 diester of fatty acid |
| COP) | |
| SURFACT/DEF (ALCOHOL | ETHOXYLATED |
| ALKOXYLATES) | LINEAR ALCOHOL |
| | C12-15, 7-MOL |
| SURFACT/DEF (EO-PO BLOCK | PLURONIC F108 |
| COP) | |
| SURFACT/DEF (ALCOHOL | LUTENSOL XL50 |
| ALKOXYLATES) | |
| SURFACT/DEF (ALCOHOL | LUTENSOL XL 80 |
| ALKOXYLATES) | |
| SURFACT/DEF (EO-PO BLOCK | 25R 2 diester of fatty acid |
| COP) | |
| SURFACT/DEF (ALCOHOL | ISO-TDA 7 |
| ALKOXYLATES) | |
| SURFACT/DEF (ALCOHOL | MULTISO 13/120 |
| ALKOXYLATES) | |
| SURFACT/DEF (ALCOHOL | ISO-TDA 6 |
| ALKOXYLATES) | |
| SURFACT/DEF (ALCOHOL | LUTENSOL ON 60 |
| ALKOXYLATES) | |
| SURFACT/DEF (ALCOHOL | LUTENSOL ON 60 |
| ALKOXYLATES) | |
| SURFACT/DEF (ALCOHOL | ETHOXYLATED |
| ALKOXYLATES) | LAURYL ALCOHOL (10 |
| | MOLES EO) |
| SURFACT/DEF (ALCOHOL | LINEAR ALCOHOL C |
| ALKOXYLATES) | 9-11, 6 MOLES EO |
| SURFACT/DEF (ALCOHOL | LUTENSOL ON 60 |
| ALKOXYLATES) | |
| SURFACT/DEF (ALCOHOL | LUTENSOL ON 60 |
| ALKOXYLATES) | |
| SURFACT/DEF (ALCOHOL | LUTENSOL ON 60 |
| ALKOXYLATES) | |
| SURFACT/DEF (ALCOHOL | LUTENSOL XP 80 |
| ALKOXYLATES) | |
| SURFACT/DEF (ALCOHOL | ETHOXY LATED (2.5- |
| ALKOXYLATES) | MOLE) SODIUM |
| | LAURYL ETH |
| SURFACT/DEF (ALCOHOL | NEODOL 91-6 |
| ALKOXYLATES) | |
| SURFACT/DEF (ALCOHOL | NEODOL 91-6 |
| ALKOXYLATES) | |
| SURFACT/DEF (ALCOHOL | NEODOL 91-6 |
| ALKOXYLATES) | |
| SURFACT/DEF (ALCOHOL | NEODOL 91-6 |
| ALKOXYLATES) | |
| SURFACT/DEF (EO-PO BLOCK | PLURONIC F108 |
| COP) | |
| SURFACT/DEF (EO-PO BLOCK | PLURONIC F108 |
| COP) | |
| SURFACT/DEF (EO-PO BLOCK | PLURONIC F108 |
| COP) | |
| SURFACT/DEF (EO-PO BLOCK | PLURONIC F108 |
| COP) | |
| SURFACT/DEF (EO-PO BLOCK | EO/PO BLOCK |
| COP) | COPOLYMER PE 6200 |
| SURFACT/DEF (EO-PO BLOCK | EO/PO BLOCK |
| COP) | COPOLYMER PE 6100 |
| SURFACT/DEF (ALCOHOL | PRIMARY ALCOHOL |
| ALKOXYLATES) | ETHOXY LATE 23-9 |
| SURFACT/DEF (ALCOHOL | LAURYL ALCOHOL |
| ALKOXYLATES) | ETHOXY LATE (9 |
| | MOLES EO) 1 |
| SURFACT/DEF (ALCOHOL | PRIMARY ALCOHOL |
| ALKOXYLATES) | ETHOXYLATE 23-9 |
| SURFACT/DEF (ALCOHOL | PRIMARY ALCOHOL |
| ALKOXYLATES) | ETHOXYLATE 23-7 |
| SURFACT/DEF (ALCOHOL | ETHOXY LATED (3- |
| ALKOXYLATES) | MOLE) SODIUM |
| | LAURYL ETHER |
| SURFACT/DEF (ALCOHOL | ETHOXYLATED |
| ALKOXYLATES) | SECONDARY |
| | ALCOHOL C11-15, 40 |
| SURFACT/DEF (EO-PO BLOCK | SYNPERONIC PE/F 108- |
| COP) | FL-NAA-(CQ) |
| SURFACT/DEF (EO-PO BLOCK | SYNPERONIC 13/10 |
| COP) | |
| SURFACT/DEF (EO-PO BLOCK | SYNPERONIC PE/F 108- |
| COP) | FL-NAA-(CQ) |
| SURFACT/DEF (ALCOHOL | TDA-5 |
| ALKOXYLATES) | |
| SURFACT/DEF (ALCOHOL | TDA-5 |
| ALKOXYLATES) | |
| SURFACT/DEF (ALCOHOL | T-DET A 136 |
| ALKOXYLATES) | |
| SURFACT/DEF (ALCOHOL | T-DET A 269 (T-DET |
| ALKOXYLATES) | 1216-9) |
| SURFACT/DEF (EO-PO BLOCK | TERGITOL 15-S-40 |
| COP) | |
| SURFACT/DEF (EO-PO BLOCK | TERGITOL 15-S-20 80% |
| COP) | |
| SURFACT/DEF (ALCOHOL | PRIMARY ALCOHOL |
| ALKOXYLATES) | ETHOXY LATE 23-7 |
| SURFACT/DEF (ALCOHOL | PRIMARY ALCOHOL |
| ALKOXYLATES) | ETHOXY LATE 23-9 |
| SURFACT/DEF (ALCOHOL | PRIMARY ALCOHOL |
| ALKOXYLATES) | ETHOXYLATE 23-9 |
| SURFACT/DEF (EO-PO BLOCK | TERGITOL XD |
| COP) | |
| SURFACT/DEF (EO-PO BLOCK | TERGITOL XD |
| COP) | |
| SURFACT/DEF (EO-PO BLOCK | TERGITOL XD |
| COP) | |
| SURFACT/DEF (EO-PO BLOCK | TERGITOL XD |
| COP) | |
| SURFACT/DEF (ALCOHOL | TOMADOL 1-5 |
| ALKOXYLATES) | |
| SURFACT/DEF (ALCOHOL | TOMADOL 1-5 |
| ALKOXYLATES) | |
| SURFACT/DEF (ALCOHOL | TOMADOL 1-7 |
| ALKOXYLATES) | |
| SURFACT/DEF (ALCOHOL | TOMADOL 1-7 |
| ALKOXYLATES) | |
| SURFACT/DEF (ALCOHOL | TOMADOL 1-9 |
| ALKOXYLATES) | |
| SURFACT/DEF (EO-PO BLOCK | TOXIMUL 8320 |
| COP) | |
| SURFACT/DEF (EO-PO BLOCK | TOXIMUL 8320 |
| COP) | |
| SURFACT/DEF (EO-PO BLOCK | TOXIMUL 8320 |
| COP) | |
| SURFACT/DEF (ALCOHOL | ISO-TDA 5 |
| ALKOXYLATES) | |

The non-ionic surfactant(s) can be present in an amount of from 2 wt % to 35 wt %, or from 5 wt % to 35 wt %, or from 2 wt % to 30 wt %, or from 2 wt % to 25 wt %, or from 2 wt % to 20 wt % or more, from 3 wt % to 20 wt %, from 5 wt % to 20 wt %, from 10 wt % to 20 wt %, from 15 wt % to 20 wt %, from 2 wt % to 15 wt %, from 2 wt % to 10 wt %, from 2 wt % to 8 wt %, from 2 wt % to 5 wt %, or any range based upon any two values described herein based on total weight of the dehairing formulation.

The divalent salt can comprise, consists of, or include calcium salt. Examples of divalent salts include, but are not limited to, where the divalent cation is magnesium, zinc, barium, iron, calcium, or nickel, or any combinations thereof.

The divalent salt can be present in an amount of from 0.1 wt % to 1 wt % or more, from 0.2 wt % to 1 wt %, from 0.3 wt % to 1 wt %, from 0.5 wt % to 1 wt %, from 0.7 wt % to 1 wt %, from 0.1 wt % to 0.9 wt %, from 0.1 wt % to 0.8 wt %, from 0.1 wt % to 0.7 wt %, from 0.1 wt % to 0.6 wt %, from 0.1 wt % to 0.5 wt %, or any range based upon any two values described herein based on total weight of the dehairing formulation.

The calcium hydroxide (or lime) utilized in the method can be present in an amount of from about 0.01 wt % to about 1 wt %, based on the total wt % of the animal skins or hides prior to the soaking step. This amount can be from 0.01 wt % to 1 wt %, from 0.03 wt % to 1 wt %, from 0.05 wt % to 1 wt %, from 0.07 wt % to 1 wt %, from 0.1 wt % to 1 wt %, from 0.3 wt % to 1 wt %, from 0.5 wt % to 1 wt %, from 0.7 wt % to 1 wt %, from 0.01 wt % to 0.9 wt %, from 0.01 wt % to 0.8 wt %, from 0.01 wt % to 0.7 wt %, from 0.01 wt % to 0.6 wt %, from 0.01 wt % to 0.6 wt %, from 0.01 wt % to 0.5 wt %, from 0.01 wt % to 0.4 wt %, from 0.01 wt % to 0.3 wt %, from 0.01 wt % to 0.2 wt %, from 0.01 wt % to 0.1 wt %, or any range based upon any two values described herein.

In the present invention, it has been discovered that better results with respect to dehairing of the skin or hide can be achieved when the calcium hydroxide contacts the animal skins or hides within about 1 hour (such as within 50 minutes or within 40 minutes, or within 30 minutes, or within 20 minutes, or within 10 minutes, or within 5 minutes, or within 3 minutes, or within 1 minute, or within 30 seconds, or within 10 seconds, or simultaneously) of the dehairing formulation doing so. The calcium hydroxide can optionally be introduced first or the dehairing formulation can be introduced first or both the calcium hydroxide and the dehairing formulation can be added at the same time to the drum containing the soaked skins or hides. Optionally, the calcium hydroxide can be pre-combined with the dehairing formulation and the combined solution introduced to the drum. The calcium hydroxide can be added in solid form or dissolved in water before being added to the drum.

When the dehairing formulation is present as a concentrate, optionally, the concentrate can be pre-diluted with water prior to the introduction of the dehairing formulation into the drum containing the skins or hides. The dilution rate can be any amount, for instance, a weight ratio of (dehairing formulation to water) from 1:10 to 1:30, or from 1:10 to 1:25, or from 1:10 to 1:20, or from 1:10 to 1:15, or any range based upon any two values described herein.

When the dehairing formulation comprises more than one component, the dehairing formulation can be added to the drum or containing with the skins or hides as a single formulation or as a multi-part formulation. For instance, the multi-part formulation can comprise a first part that includes the at least one carbohydrase enzyme, the at least one non-ionic surfactant, and a second part that includes at least the divalent salt. When multi-part, each part can be added at the same time or within 1 hour of each other, such as within 30 minutes, or within 15 minutes, or within 5 minutes or within 1 minute or within 30 seconds, or within 10 seconds, or within 5 seconds of each other.

The dehairing formulation can be added to the animal skins and/or hides and/or to the aqueous soaking solution as a combined formulation (e.g., a) at least one carbohydrase enzyme, b) optionally at least one lipase enzyme, c) optionally at least one non-ionic surfactant, d) optionally at least one divalent salt). Alternatively, the various individual components comprising the dehairing formulation can be added to the animal skins and/or hides and/or to the aqueous soaking solution separately or in a combination and/or in any order. For instance, the carbohydrase enzyme can be added separately and/or initially and then, if used, the lipase enzyme can be added afterwards. The non-ionic surfactant, for instance, if present, can be added along with either or both the carbohydrase enzyme and/or the lipase enzyme. The divalent salt, for instance, if present, can be added along with either or both the carbohydrase enzyme and/or the lipase enzyme. The non-ionic surfactant and/or the divalent salt, if used, can be added before any enzyme addition or in between enzyme additions and/or after enzyme additions.

In the present invention, the carbohydrase enzyme can be added prior to, at the same time as, or after the adding of any of the other components that may form the dehairing formulation (e.g., the lipase enzyme, the non-ionic surfactant, and/or the divalent salt). For instance, the carbohydrase enzyme can be added within 1 second, within 5 seconds, within 15 seconds, within 1 minute, within 5 minutes, or within 10 minutes (or other times) of the adding of the one or more other components that may form the dehairing formulation as described herein. The carbohydrase enzyme can be added as a mixture with one or more of the other components that may form the dehairing formulation (e.g., the lipase enzyme, the non-ionic surfactant, and/or the divalent salt). The carbohydrase enzyme can be added separately (e.g., by way of separate feed lines) with one or more of the other components that may form the dehairing formulation. The dehairing formulation and/or one or components forming the dehairing formulation can be added as a single dose, as multiple doses, or on a continuously or semi-continuous feed to the animal skin(s) and/or hide(s) and/or the aqueous soaking solution.

As an option, the method of the present invention, in general can be conducted in the absence of any protease enzyme.

With respect to the reducing chemical, examples include, but are not limited to, a sulfide or salt thereof, a hydrosulfide or salt thereof, a thioglycolate, an amine, or additional lime, or any combinations thereof.

The reducing chemical can be utilized in any effective amount. For instance, the reducing chemical can be utilized in an amount of from about 0.1 wt % to about 20 wt %, based on the total wt % of the animal skins or hides prior to the soaking step. The amount can be from 0.1 wt % to 20 wt %, from 0.1 wt % to 15 wt %, from 0.1 wt % to 10 wt %, from 0.1 wt % to 5 wt %, from 0.1 wt % to 2 wt %, from 0.2 wt % to 20 wt %, from 0.5 wt % to 20 wt %, from 1 wt % to 20 wt %, from 2 wt % to 20 wt %, from 5 wt % to 20 wt %, or any range based upon any two values described herein.

The at least one reducing chemical can be present in an amount of from about 0.1 wt % to about 2.5 wt % or more, based on the total wt % of the animal skins or hides prior to the soaking step. This amount can be from 0.1 wt % to 2.5 wt %, or from 0.1 wt % to 2.25 wt %, or from 0.1 wt % to 2 wt %, or from 0.1 wt % to 1.75 wt %, or from 0.1 wt % to 1.5 wt %, or from 0.1 wt % to 1.5 wt %, or from 0.1 wt % or from 1.25 wt %, or from 0.1 wt % to 1 wt %, or from 0.1 wt % to 0.75 wt %, or from 0.1 wt % to 0.5 wt %, or from 0.1 wt % to 0.25 wt %, or from 0.2 wt % to 2.5 wt %, or from 0.3 wt % to 2.5 wt %, or from 0.5 wt % to 2.5 wt %, or from 0.75 wt % to 2.5 wt %, or any range based upon any two values described herein.

The reducing chemical(s) can be added after the dehairing formulation and/or the calcium hydroxide are added. Generally, the reducing chemical(s) is added afterwards. For instance, the reducing chemical(s) can be added within minutes to hours of dehairing formulation and/or calcium hydroxide being added to the drum or container for the dehairing step. For instance, the reducing chemical(s) can be added within about 1 hour to 12 hours of the dehairing formulation being added (such as within 1 hour, within 2 hours, within 3 hours, within 4 hours, within 5 hours, within 6 hours, within 7 hours, within 8 hours, within 9 hours, within 10 hours, within 11 hours, within 50 minutes or within 40 minutes, or within 30 minutes, or within 20 minutes, or within 10 minutes, or within 5 minutes, or within 3 minutes, or within 1 minute, or within 30 seconds, or within 10 seconds, or simultaneously) of the dehairing formulation being added.

When the dehairing formulation is contacting the skins or hides, for instance in the bath within the drum, the pH of the bath can be and preferably is from about 8 to about 13. This pH can be present before the dehairing formulation is added or introduced or this pH can be adjusted to be in this pH range after the dehairing formulation is added, such as within 1 hour of the dehairing formulation being added (e.g., within 45 minutes, within 30 minutes, within 15 minutes, within 10 minutes, within 5 minutes, within 1 minute, or within 30 seconds or short period of time).

The step of contacting or floating the skins or hides with the dehairing formulation occurs for a time of from about 2 hours to 24 hours, or more. This contact time can be from 2 hours to 24 hours, from 2 hours to 20 hours, from 2 hours to 15 hours, from 2 hours to 10 hours, from 2 hours to 5 hours, from 2 hours to 3 hours, from 5 hours to 24 hours, from 7 hours to 24 hours, from 10 hours to 24 hours, from 12 hours to 24 hours, from 15 hours to 24 hours, or any range based upon any two values described herein.

The contacting with the dehairing formulation, for instance, can occur for a time of from about 2 hours to 24 hours at a temperature of from about 15 deg C. to 50 deg C. (e.g., from 15 deg C. to 45 deg C., from 15 deg C. to 40 deg C., from 15 deg C. to 30 deg C., from 15 deg C. to 25 deg C.).

The above timing and/or temperature apply as well once the calcium hydroxide and the reducing chemical(s) are present.

After the dehairing step, as an option, subsequent steps can be used on the dehaired skins or hides that are conventional in the leather or tannery industry. For instance, one or more of the following steps can be conducted afterwards: a deliming process, a fleshing process, a splitting process, a bating process, a pickling process and/or a tanning process and optionally followed by a basification process.

When skins and/or hides are processed, many times, the hides and/or skins are provided to the tanneries as salted or dried raw hides or skins. The processing of hides or skins into leather typically comprises several different process steps including the steps of soaking, unhairing, liming, deliming, bating, pickling and tanning. These steps constitute the wet processing and are performed in the beamhouse. Processes for the manufacture of leather are well known to the person skilled in the art and have been described, for instance, in WO 94/06942, WO 90/12118, U.S. Pat. No. 3,840,433, EP505920A1, GB 2233665A 1 and U.S. Pat. No. 3,986,926, all incorporated in their entirety by reference herein. Any one or more steps or components described in any one of these documents can be utilized in the methods of the present invention as long as the step and/or component does not conflict with the use of the dehairing formulation of the present invention.

The present invention, in part, further relates to a dehairing formulation. The dehairing formulation can be a concentrate or be a dilution. When a concentrate (or when not including water or a solvent in the wt %), the dehairing formulation can comprise or consists of or include from about 10 wt % to 90 wt % carbohydrase enzyme, optionally lipase enzyme (e.g., from about 0.1 wt % to about 50 wt %), from about 2 wt % to 20 wt % non-ionic surfactant; and from about 0.1 wt % to 1 wt % divalent salt, based on total weight of the dehairing formulation. The various weight % ranges (and sub-ranges) provided earlier for each component above are applicable here.

As an option, the dehairing formulation can contain at least one solvent. The solvent(s) can be an aqueous based solvent. The solvent can be, for instance, water, ethylene glycol, propylene glycol, ethylene diglycol, methylene diglycol, or any combinations thereof. The solvent can be present, as an example, in an amount of from about 10 wt % to 70 wt % based on the total weight of the dehairing formulation. The solvent amount can be from 10 wt % to 60 wt %, from 10 wt % to 50 wt %, from 10 wt % to 40 wt %, from 10 wt % to 30 wt %, from 10 wt % to 20 wt %, from 15 wt % to 70 wt %, from 20 wt % to 70 wt %, from 25 wt % to 70 wt %, from 30 wt % to 70 wt %, from 35 wt % to 70 wt %, from 40 wt % to 70 wt %, from 45 wt % to 70 wt %, from 50 wt % to 70 wt %, from 55 wt % to 70 wt %, or any range based upon any two values described herein.

The step of “contacting” the hide or skin with the dehairing formulation described herein can comprise, consists essentially of, consist of, or include contacting the hide or skin with the dehairing formulation of the present invention. The contacting can be by spraying of the formulation onto a surface(s) of the hide or skin, soaking the hide or skin with the formulation of the present invention, coating the hide or skin with the formulation, dipping of the hide or skin into a vat that contains the formulation of the present invention, and the like. Essentially, any means can be used to ensure that one or all surfaces or substantially all surfaces of the hide or skin are contacted (e.g., coated, submerged, enveloped) with the formulation of the present invention.

With the present invention, the methods of the present invention and/or the dehairing formulation, and in particular, the dehairing process can improve the removal of hair, hair roots and/or epidermis keratin.

With the present invention, the methods of the present invention and/or the dehairing formulation, and in particular, the dehairing process can provide effective dehairing of the skins and hides and also improve the leather grain quality.

With the present invention, the methods of the present invention and/or the dehairing formulation, and in particular, the dehairing process can provide a dehairing process that can use enzymatic treatment but in the absence of protease enzymes.

With the present invention, the methods of the present invention and/or the dehairing formulation, and in particular, the dehairing process can provide a dehairing process that can reduce the amount of lime utilized in the dehairing process. For instance, the amount of lime can be reduced by at least 5 wt %, by at least 10 wt %, by at least 25 wt % compared to same process without using the dehairing formulation of the present invention but just lime and achieving about the same degree of dehairing.

With the present invention, the methods of the present invention and/or the dehairing formulation, and in particular, the dehairing process can provide a dehairing process that can reduce the amount of time for effective dehairing, especially utilizing an enzyme treatment.

With the present invention, the methods of the present invention and/or the dehairing formulation, and in particular, the dehairing process can provide an enzymatic dehairing process that can confine the enzyme activity to the epidermis layer, hair follicles/hair and basement membrane of the skins and hides.

With the present invention, the methods of the present invention and/or the dehairing formulation, and in particular, the dehairing process can provide an enzymatic dehairing process that can greatly diminish or avoid the extension of enzyme or proteolytic activity towards the collagen structure of the skins and hides.

With the present invention, the methods of the present invention and/or the dehairing formulation, and in particular, the dehairing process can provide a dehairing process that can provide a dehaired skin or hide that is has well defined pores and/or flatter grains and/or have improved characteristics of better cleaning and/or better uniformity of the softness of the pelt and in the final leather, and obtain a clearer pelt, which provides a more vibrant and homogeneous dyeing of the pelt, and which can improve the process with some reduction in bleaching agents.

The following examples are intended to illustrate, not limit, the present invention. In the following examples, all parts are proportions by weight unless otherwise specified.

### EXAMPLES

### Example 1

This example demonstrates the superior results obtainable using the methods and formulations of the present invention.

Formulations were prepared based on the following:

At least one solvent selected from the following in an amount of from 10 wt % to 70 wt % (based on the total weight of the dehairing composition): water, ethylene glycol, propylene glycol, ethylene diglycol, and/or methylene diglycol.

A blend of surfactants having an HLB of from 8 to 24 and selected from the following and in an amount of from 5 wt % to 35 wt % (based on the total weight of the dehairing composition): blend of PLURONIC F108 and SYNPERONIC PE/F108; blend of TOMADOL 1-7, ALKONAT L70, and SY NPERONIC 13/10; blend of TOXIMUL 8320, TERGITOL XD, and ATLAS 5000G; blend of ISO-TDA-60, Lutensol ON 60, and ISO-TDA-70; blend of PEG-400, PEG-400 DIOLATE, PEG-600, PEG-600CDIOLATE, ALKONAT L90; ALKONAT L100, ETOXYLATED LAURYL ESTER SULFATE 9E0, and ETHOXYLATED LAURYL ALCOHOL (10 MOLES EO).

At least one carbohydrase selected from the following in an amount of from 10 wt % to 60 wt % (based on the total weight of the dehairing composition): xylanase, cellulase, amylase, and/or mannanase.

### EXAMPLES OF FORMULATIONS

### Example 1A

| Raw material | wt % |
|---|---|
| Water | 50 |
| Ethylene glycol | 10 |
| Tomadol 1-7 | 8 |
| PEG 400 | 8 |
| Pluronic F108 | 14 |
| Cellulase | 10 |

### Example 1B

| Raw material | wt % |
|---|---|
| M ethylene diglycol | 5 |
| ATLAS 5000G | 15 |
| Lutensol ON 60 | 8 |
| Alkonat L 90 | 10 |
| Water | 37 |
| X ylanase | 25 |

### Example 1C

| Raw material | wt % |
|---|---|
| Synperonic | 2 |
| PE/F108 | |
| PEG 600 | 3 |
| M annanase | 42 |
| Tergitol XD | 5 |
| Water | 39 |
| Ethylene Diglycol | 9 |

### Example 1D

| Raw material | wt % |
|---|---|
| Alkonat L100 | 12 |
| ISO TDA 60 | 8 |
| Toximul 8320 | 2 |
| Amylase | 28 |
| Ethylene glycol | 9 |
| Water | 41 |

A dehairing formulation was prepared by mixing together the following components and evaluated in the methods described below: 50 to 70 wt % water, a blend of two surfactants with an HLB of 8 to 24 that are an oleo (fatty acid) ester in an amount of 10 wt % to 30 wt %, and an amylase enzyme in an amount of 25 to 50 wt %. A 0.4 gram sample of this dehairing formulation was combined with 50 grams of water. Lime in an amount of 0.25 gram was then added after the dehairing formulation was added.

In this example, 30 grams samples of a hide with hair on the surface were used. The hides were subjected to a soaking step for about 1 hour using 300 wt % water, 0.3 wt % degreaser, and 0.2 wt % bactericide, where the wt % is based on the total weight of green hide weight. The pH of the soaking solution was about 9.1 to 9.7 and the temperature of the soaking solution was about 25 deg C. The soaking solution was then drained. After soaking, the soaked hides then subjected to a dehairing step or a control, wherein the soaked hides were immersed in either a) water with a dehairing formulation of the present invention that contained a carbohydrase enzyme and non-ionic surfactant(s) and lime, or b) water with a carbohydrase enzyme and lime (no surfactant) (of the present invention as well), or c) a control where lime with water was present (no enzyme and no surfactant). The immersion in one of a), b), or c) was for 4 hours. Then the condition of the hide was evaluated. The hides immersed in c) kept all of the hair on the hide. The hides immersed in b) had approximately 50% to 60% of the hair removed. The hides immersed in a) had on average approximately 90% to 99% of the hair removed. A mount of hair removed is based on surface area. Thus, the examples showed that a carbohydrase enzyme can remove hair in a dehairing step and that a combination of carbohydrase enzyme with non-ionic surfactant is surprisingly more effective as the surfactant appears to enhance the ability of the carbohydrase enzyme's ability to remove/dissolve hair from the hide. The pH during the dehairing step started at about 12 pH and after 4 hours was about 11.5 pH, and the temperature of the dehairing solution or control was about 27 to 30 deg C.

The present invention includes the following aspects/embodiments/features in any order and/or in any combination:
1. The present invention relates to a method for dehairing animal skins or hides, said method comprising soaking animal skins or hides having skin hair in an aqueous soaking solution to wet the animal skins or hides having skin hair and obtain wet skins or hides. contacting the wet skins or hides with a dehairing formulation and calcium hydroxide for a sufficient time so as to decompose at least a majority of the skin hair present on the wet skins or hides, wherein said dehairing formulation comprises at least one carbohydrase enzyme; contacting the wet skins or hides while the dehairing formulation is present with at least one reducing chemical; and recovering the animal skins or hides.
2. The method of any preceding or following embodiment/feature/aspect, wherein the dehairing formulation further comprises at least one non-ionic surfactant and/or at least one divalent salt and/or at least one lipase enzyme.
3. The method of any preceding or following embodiment/feature/aspect, wherein the aqueous soaking solution comprises primarily water and said soaking is from about 1 hour to 20 hours.
4. The method of any preceding or following embodiment/feature/aspect, wherein said contacting of the wet skins or hides with the dehairing formulation comprises floating the wet skins or hides in a bath that contains at least the dehairing formulation and water.
5. The method of any preceding or following embodiment/feature/aspect, wherein the dehairing formulation is present in the bath in an amount of from about 0.1 wt % to about 1 wt %, based on the total wt % of the animal skins or hides prior to said soaking.
6. The method of any preceding or following embodiment/feature/aspect, wherein the dehairing formulation, as a concentrate, comprises from about 10 wt % to 90 wt % carbohydrase enzyme, based on total weight of the dehairing formulation.
7. The method of any preceding or following embodiment/feature/aspect, wherein the dehairing formulation, as a concentrate, comprises from about 10 wt % to 90 wt % carbohydrase enzyme, from about 2 wt % to 20 wt % non-ionic surfactant; and from about 0.1 wt % to 1 wt % divalent salt, based on total weight of the dehairing formulation.
8. The method of any preceding or following embodiment/feature/aspect, wherein said calcium hydroxide is present in an amount of from about 0.01 wt % to about 1 wt %, based on the total wt % of the animal skins or hides prior to said soaking.
9. The method of any preceding or following embodiment/feature/aspect, wherein the calcium hydroxide is contacts the animal skins or hides within about 1 hour of the dehairing formulation doing so.
10. The method of any preceding or following embodiment/feature/aspect, wherein the dehairing formulation is added to a drum as a single formulation.
11. The method of any preceding or following embodiment/feature/aspect, wherein the dehairing formulation is added to a drum as a multi-part formulation.
12. The method of any preceding or following embodiment/feature/aspect, wherein the multi-part formulation comprises a first part that includes the at least one carbohydrase enzyme, the at least one non-ionic surfactant, and a second part that includes at least the divalent salt.
13. The method of any preceding or following embodiment/feature/aspect, wherein the soaking is in the absence of any enzyme.
14. The method of any preceding or following embodiment/feature/aspect, wherein the dehairing formulation in the absence of any protease enzyme.
15. The method of any preceding or following embodiment/feature/aspect, wherein said method is in the absence of any protease enzyme.
16. The method of any preceding or following embodiment/feature/aspect, wherein the at least one reducing chemical is a sulfide or salt thereof, a hydrosulfide or salt thereof, a thioglycolate, an amine, or additional lime, or any combinations thereof.
17. The method of any preceding or following embodiment/feature/aspect, wherein the at least one reducing chemical is present in an amount of from about 0.1 wt % to about 2.5 wt %, based on the total wt % of the animal skins or hides prior to said soaking.
18. The method of any preceding or following embodiment/feature/aspect, wherein the carbohydrase enzyme consists of one or more microbial enzymes.
19. The method of any preceding or following embodiment/feature/aspect, wherein the contacting with the dehairing formulation occurs at a pH of from about 8 to about 13.
20. The method of any preceding or following embodiment/feature/aspect, wherein the contacting with the dehairing formulation occurs for a time of from about 2 hours to 24 hours.
21. The method of any preceding or following embodiment/feature/aspect, wherein the soaking removes no or essentially no skin hair from the animal hides or skins.
22. The method of any preceding or following embodiment/feature/aspect, wherein the divalent salt comprises calcium salt.
23. The method of any preceding or following embodiment/feature/aspect, wherein the divalent salt comprises a divalent cation that is magnesium, zinc, barium, iron, calcium, or nickel, or any combinations thereof.
24. The method of any preceding or following embodiment/feature/aspect, wherein said method further comprises the following steps: a deliming process, a bating process, a pickling process and a tanning process and optionally followed by a basification process.
25. The method of any preceding or following embodiment/feature/aspect, wherein the contacting with the dehairing formulation occurs for a time of from about 2 hours to 24 hours at a temperature of from about 15 deg C. to 50 deg C.
26. The method of any preceding or following embodiment/feature/aspect, wherein the carbohydrase enzyme has a specific enzyme activity of from about 1,000 to 100,000, where said specific enzyme activity is based on an enzyme per milligram of total protein, expressed in μmol min⁻¹ mg⁻¹.
27. The method of any preceding or following embodiment/feature/aspect, wherein the reducing chemical is utilized in an amount of from about 0.1 wt % to about 20 wt %, based on the total wt % of the animal skins or hides prior to said soaking.
28. The method of any preceding or following embodiment/feature/aspect, wherein the non-ionic surfactant is one or more of the following: an oleo (fatty acid) ester, an oleo alkoxylate, an oleo fatty acid oil, an alcohol alkoxylate, and EO-PO block copolymer, or any combinations or blends thereof.
29. The method of any preceding or following embodiment/feature/aspect, wherein the at least one carbohydrase enzyme is a xylanase, a mannanase, a cellulase, and/or an amylase.
30. The method of any preceding or following embodiment/feature/aspect, wherein the dehairing formulation and calcium hydroxide are added within 1 hour of each other.
31. The method of any preceding or following embodiment/feature/aspect, wherein the dehairing formulation is added first before any other component after said soaking.
32. The method of any preceding or following embodiment/feature/aspect, wherein the dehairing formulation utilized in the contacting is a dilution of the dehairing formulation in water, at a weight ratio of 1:10 (dehairing formulation: water) to 1:30 (dehairing formulation: water).
33. The present invention further relates to a dehairing formulation comprising from about 10 wt % to 90 wt % carbohydrase enzyme, from about 2 wt % to 20 wt % non-ionic surfactant; and from about 0.1 wt % to 1 wt % divalent salt, based on total weight of the dehairing formulation.
34. The method of any preceding or following embodiment/feature/aspect, wherein the dehairing formulation further comprises at least one lipase enzyme, non-ionic surfactant and/or at least one divalent salt.
35. The method of any preceding or following embodiment/feature/aspect, wherein the dehairing formulation further comprises at least one lipase enzyme.
36. The present invention further relates to a dehairing formulation comprising from about 10 wt % to 90 wt % carbohydrase enzyme, from about 0.1 wt % to about 50 wt % lipase enzyme, from about 2 wt % to 20 wt % non-ionic surfactant; and from about 0.1 wt % to 1 wt % divalent salt, based on total weight of the dehairing formulation.
37. The method of any preceding or following embodiment/feature/aspect, wherein the dehairing formulation, as a concentrate, comprises from about 0.1 wt % to 50 wt % lipase enzyme, based on total weight of the dehairing formulation.
38. The method of any preceding or following embodiment/feature/aspect, wherein the dehairing formulation, as a concentrate, comprises from about 10 wt % to 90 wt % carbohydrase enzyme, from about 0.1 wt % to about 50 wt % lipase enzyme, from about 2 wt % to 20 wt % non-ionic surfactant; and from about 0.1 wt % to 1 wt % divalent salt, based on total weight of the dehairing formulation.
39. The dehairing formulation of any preceding or following embodiment/feature/aspect, where the formulation has any one or more of the features or attributes described above in the method embodiments.

The present invention can include any combination of these various aspects, features, or embodiments above and/or below as set forth in sentences and/or paragraphs. Any combination of disclosed features herein is considered part of the present invention and no limitation is intended with respect to combinable features.

Applicants specifically incorporate the entire contents of all cited references in this disclosure. Further, when an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the invention be limited to the specific values recited when defining a range.

It will be apparent to those skilled in the art that various modifications and variations can be made to the embodiments of the present invention without departing from the spirit or scope of the present invention. Thus, it is intended that the present invention covers other modifications and variations of this invention provided they come within the scope of the appended claims and their equivalents.

What is claimed is:

1. A method for dehairing animal skins or hides, said method comprising soaking animal skins or hides having skin hair in an aqueous soaking solution to wet the animal skins or hides having skin hair and obtain wet skins or hides;
contacting the wet skins or hides with a dehairing formulation and calcium hydroxide for a sufficient time so as to decompose at least a majority of the skin hair present on the wet skins or hides, wherein said dehairing formulation comprises at least one carbohydrase enzyme;
contacting the wet skins or hides while the dehairing formulation is present with at least one reducing chemical; and recovering the animal skins or hides.

2. The method of claim 1, wherein the dehairing formulation further comprises at least one non-ionic surfactant and/or at least one divalent salt and/or at least one lipase enzyme.

3. The method of claim 1, wherein the aqueous soaking solution comprises primarily water and said soaking is from about 1 hour to 20 hours.

4. The method of claim 1, wherein said contacting of the wet skins or hides with the dehairing formulation comprises floating the wet skins or hides in a bath that contains at least the dehairing formulation and water.

5. The method of claim 1, wherein the dehairing formulation is present in the bath in an amount of from about 0.1 wt % to about 1 wt %, based on the total wt % of the animal skins or hides prior to said soaking.

6. The method of claim 1, wherein the dehairing formulation, as a concentrate, comprises from about 10 wt % to 90 wt % carbohydrase enzyme, based on total weight of the dehairing formulation.

7. The method of claim 1, wherein the dehairing formulation, as a concentrate, comprises from about 10 wt % to 90 wt % carbohydrase enzyme, from about 2 wt % to 20 wt % non-ionic surfactant; and from about 0.1 wt % to 1 wt % divalent salt, based on total weight of the dehairing formulation.

8. The method of claim 1, wherein said calcium hydroxide is present in an amount of from about 0.01 wt % to about 1 wt %, based on the total wt % of the animal skins or hides prior to said soaking.

9. The method of claim 1, wherein the calcium hydroxide is contacts the animal skins or hides within about 1 hour of the dehairing formulation doing so.

10. The method of claim 1, wherein the dehairing formulation is added to a drum as a single formulation.

11. The method of claim 1, wherein the dehairing formulation is added to a drum as a multi-part formulation.

12. The method of claim 1, wherein the multi-part formulation comprises a first part that includes the at least one carbohydrase enzyme, the at least one non-ionic surfactant, and a second part that includes at least the divalent salt.

13. The method of claim 1, wherein the soaking is in the absence of any enzyme.

14. The method of claim 1, wherein the dehairing formulation in the absence of any protease enzyme.

15. The method of claim 1, wherein said method is in the absence of any protease enzyme.

16. The method of claim 1, wherein the at least one reducing chemical is a sulfide or salt thereof, a hydrosulfide or salt thereof, a thioglycolate, an amine, or additional lime, or any combinations thereof.

17. The method of claim 1, wherein the at least one reducing chemical is present in an amount of from about 0.1 wt % to about 2.5 wt %, based on the total wt % of the animal skins or hides prior to said soaking.

18. The method of claim 1, wherein the carbohydrase enzyme consists of one or more microbial enzymes.

19. The method of claim 1, wherein the contacting with the dehairing formulation occurs at a pH of from about 8 to about 13.

20. The method of claim 1, wherein the contacting with the dehairing formulation occurs for a time of from about 2 hours to 24 hours.

21. The method of claim 1, wherein the soaking removes no or essentially no skin hair from the animal hides or skins.

22. The method of claim 1, wherein the divalent salt comprises calcium salt.

23. The method of claim 1, wherein the divalent salt comprises a divalent cation that is magnesium, zinc, barium, iron, calcium, or nickel, or any combinations thereof.

24. The method of claim 1, wherein said method further comprises the following steps: a deliming process, a bating process, a pickling process and a tanning process and optionally followed by a basification process.

25. The method of claim 1, wherein the contacting with the dehairing formulation occurs for a time of from about 2 hours to 24 hours at a temperature of from about 15 deg C. to 50 deg C.

26. The method of claim 1, wherein the carbohydrase enzyme has a specific enzyme activity of from about 1,000 to 100,000, where said specific enzyme activity is based on an enzyme per milligram of total protein, expressed in μmol min⁻¹ mg⁻¹.

27. The method of claim 1, wherein the reducing chemical is utilized in an amount of from about 0.1 wt % to about 20 wt %, based on the total wt % of the animal skins or hides prior to said soaking.

28. The method of claim 1, wherein the non-ionic surfactant is one or more of the following:
an oleo (fatty acid) ester, an oleo alkoxylate, an oleo fatty acid oil, an alcohol alkoxylate, and EO-PO block copolymer, or any combinations or blends thereof.

29. The method of claim 1, wherein the at least one carbohydrase enzyme is a xylanase, a mannanase, a cellulase, and/or an amylase.

30. The method of claim 1, wherein the dehairing formulation utilized in the contacting is a dilution of the dehairing formulation in water, at a weight ratio of 1:10 (dehairing formulation: water) to 1:30 (dehairing formulation: water).

31. The method of claim 1, wherein the dehairing formulation and calcium hydroxide are added within 1 hour of each other.

32. The method of claim 1, wherein the dehairing formulation is added first before any other component after said soaking.

33. A dehairing formulation comprising from about 10 wt % to 90 wt % carbohydrase enzyme, from about 2 wt % to 20 wt % non-ionic surfactant, from 0.1 wt % to 50 wt % lipase enzyme, and from about 0.1 wt % to 1 wt % divalent salt, based on total weight of the dehairing formulation.
